(19) United States
(12) Patent Application Publication
(10) Pub. No.: US 2008/0206138 A1
(43) Pub. Date: Aug. 28, 2008

(54) Radiolabelled Phenylethyl Imidazole Carboxylic Acid Ester Derivatives

(75) Inventors: Ilse Zolle, Wien (AT); Friedrich Hammerschmidt, Wien (AT)

Correspondence Address: LERNER GREENBERG STEMER LLP, P O BOX 2480, HOLLYWOOD, FL, 33022-2480, US

(73) Assignee: Ilse Zolle, Vienna (AT)

(21) Appl. No.: 12/049,835

(22) Filed: Mar. 17, 2008

Related U.S. Application Data
(63) Continuation-in-part of application No. 11/582,073, filed on Oct. 17, 2006, now Pat. No. 7,358,369.
Application No. 11/582,073 is a division of application No. 10/635,294, filed on Aug. 6, 2003, now Pat. No. 7,189,859.

(51) Int. Cl. A61K 51/04; C07D 233/90

(52) U.S. Cl. 424/1.85; 548/334.5

(57) ABSTRACT

Compounds derived from phenylethyl imidazole carboxylic acid esters have shown selective accumulation of radioactivity in the adrenal cortex, when labeled with a radioactive halogen. In particular, these compounds bind selectively to adrenocortical tissue facilitating the diagnosis of adrenal cortical masses such as incidentaloma, adenoma, primary and metastatic cortical carcinoma. Trace amounts are injected intravenously and accumulate rapidly in the adrenals, maintaining a high radioactivity plateau, which permits external imaging using computed SPECT (single photon emission) or PET (positron emission) techniques.

Independent of the position and type of the radioactive label, the compounds according to the invention are potent inhibitors of steroid P450c11 hydroxylation and bind with high affinity on sites of cortisol secretion. In order to avoid saturation of receptor sites, high specific activity labeling is mandatory for application in patients. The compounds in accordance with the invention have been found to possess an almost 1000-fold higher affinity when compared with the known, clinically used inhibitors (metyrapone, ketoconazole).

### CROSS-REFERENCE TO RELATED APPLICATIONS

This is a continuation-in-part application of copending patent application Ser. No. 11/582,073, filed Oct. 17, 2006, which was a divisional of patent application Ser. No. 10/635,294, filed Aug. 6, 2003, now U.S. Pat. No. 7,189,859 B2; the prior applications are incorporated herein by reference in their entirety.

### BACKGROUND OF THE INVENTION

### Field of the Invention

Adrenal Scintigraphy for Detection of Adrenal Cortical Pathology.

### BRIEF SUMMARY OF THE INVENTION

The invention relates to previously disclosed radioactively labelled derivatives of (R)-1-(1-phenylethyl)-1H-imidazole-5-carboxylic acid esters and methods for preparing these compounds. The invention also relates to the use of these radioactively labelled compounds as radiopharmaceuticals for functional diagnosis of adrenal disease and for therapeutic applications. In particular, these compounds bind selectively to adrenocortical tissue facilitating the diagnosis of adrenal cortical masses such as incidentaloma, adenoma, primary and metastatic cortical carcinoma.

The present invention relates to a class of substituted (R)-1-(1-phenylethyl)-1H-imidazole-5-carboxylic acid esters, which interact selectively with the mitochondrial cytochrome P-450 species in the adrenal cortex (Vanden Bossche, 1984). When labelled with a radiohalogen (iodine-123; bromine-76; fluorine-18, and others), these compounds serve as radiotracers for the diagnosis of adrenal cortical masses such as incidentaloma, adenoma, primary and metastatic cortical carcinoma. When labelled with a beta-emitting radionuclide (iodine-131; bromine-82, and others), these radiotracers may be used for radionuclide therapy.

In particular, the compounds according to this invention are potent inhibitors of steroid P450c11 hydroxylation and bind with high affinity to sites of hormone production. In fact, the compounds in accordance with this invention have been found to possess an almost 1000-fold selective affinity when compared with known, clinically used inhibitors (metyrapone, ketoconazole). Therefore, when injected intravenously, the labelled derivatives of the present invention accumulate almost exclusively in the adrenals, reaching radioactivity levels that are diagnostically useful.

The parent compound etomidate (ethyl ester; ETO) is clinically used as a short-acting hypnotic drug. When incubated with human adrenocortical tissue slices, ETO was shown to block the conversion of 11-deoxycortisol to cortisol and of 11-deoxycorticosterone (DOC) to corticosterone and aldosterone (Weber 1993; Engelhardt 1994). Metomidate (MTO), the methyl ester, is an equally potent inhibitor of steroid 11β-hydroxylation. (R)-configuration of the methyl substituent at the chiral C-atom is essential for enzyme inhibition (Vanden Bossche, 1984; Berger et al., 2002; Zolle et al., 2008).

Clinical findings with [O-methyl-¹¹C]metomidate have indicated high uptake in lesions of adrenocortical origin, including adenomas, but very low uptake in lesions of non-adrenocortical origin (Bergström 1998; 2000). Asymptomatic adrenal masses (incidentaloma) are detected incidentally by abdominal CT, and other imaging modalities. ¹¹C-metomidate showed almost 100% specificity for the identification of hormonally silent adrenocortical adenoma, when compared with CT (computed tomography) and MRI (magnetic resonance imaging). Both CT and MRI cannot differentiate silent from hormonally active adrenocortical adenoma (Bergström et al., 2000; Khan et al., 2003). However, the nature of these masses must be identified to exclude adrenocortical dysfunction, or metastatic or primary adrenal cortical cancer (Abecasis et al. 1985). While ¹¹C-metomidate specifically detects tissue of adrenocortical origin, it shows no uptake in cysts, lipoma, hematoma or metastases of other primary tumors (Khan et al., 2003).

Although ¹¹C-metomidate has “ideal” biological characteristics for scintigraphy of the adrenals and tumor derived therefrom, application of the radiopharmaceutical is limited to hospitals with a PET facility. ¹¹C is a cyclotron product and decays with a half-life of 20 min, therefore, ¹¹C-metomidate must be synthesized immediately prior to use.

Halogenations, on the other hand, offer sufficient flexibility, time for preparation and shipment. (Iodine-123 T_{1/2}=13.2 hours; Br-76 T_{1/2}=16 hours; F-18 T_{1/2}=1.8 hours). Modification of the ester function offers access to labelled R¹ derivatives, which are equally potent inhibitors (i.e. ¹¹C-MTO, ¹⁸F-FETO). Substitutions in the phenyl ring with a radiohalogen produced radiolabelled derivatives that are also disclosed in U.S. Pat. No. 7,189,859.

Enzyme inhibitors, such as metyrapone have been labelled with radioiodine for adrenal scintigraphy, however, these compounds have never been used for clinical diagnosis (Wieland, 1982; Robien & Zolle, 1983). The compounds in accordance with the present invention potently and selectively bind to adrenocortical membranes (cytochrome P-450c11).

A comparison of the binding affinities (IC₅₀ values) of some etomidate derivatives with known inhibitors clearly demonstrate the high potency of (R)-etomidate derivatives (FIG. 1). Hydrolysis of the ester function resulted in a loss of binding potency.

With the above and other objects in view there is provided, in accordance with the invention methods for clinical application of 1-(1-arylalkyl)-1H-imidazole-5-carboxylate ester derivatives of formula (I) with modified functionality R¹, R², and R³, incorporating a radioactive halogen, wherein the compound is either prepared shortly prior to administering to the subject, or prepared at least one day before the imaging is performed, and stored until needed.

In accordance with another feature of the invention, there is provided a method for performing adrenal scintigraphy for the diagnosis of associated disease, the method comprising: (a) administering to a patient an effective amount of radioactivity of a compound defined in claim 2 or 3 wherein R¹ is radioactive 2-fluoroethyl; or a compound wherein R³ is phenyl, substituted with a radioactive halogen of the following formula:

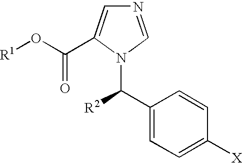

wherein R¹ and R² are each methyl, and X is radioactive iodine and wherein the compound is *I-metomidate (IMTO); or wherein R¹ is ethyl, R² is methyl, and X is radioactive iodine, wherein the compound is *I-etomidate (1-ETO), wherein the radioactive halogen is selected from the group consisting of ¹²³I, ¹²⁴I, ¹²⁵I, ¹³¹I, ⁷⁶Br, and ¹⁸F; (b) applying a suitable tomographic procedure, i.e., SPECT or PET

In accordance with yet another feature of the invention, there is provided a method for adrenal scintigraphy for the localization and characterization of abnormal adrenocortical function, wherein said associated conditions are Cushing's syndrome; primary aldosteronism; and the incidentally discovered adrenal mass; especially, adenoma; bilateralcortical nodular hyperplasia; adrenocortical carcinoma; hormonally silent adenoma.

In accordance with an added feature of the invention, there is provided a method for functional adrenal scintigraphy and diagnosis of associated disease, wherein said associated conditions are selected from the group of conditions presenting with hyperfunctioning adrenal(s), adrenocortical adenoma, and adrenal tumors, said method comprising:

diagnosis of adrenocortical adenoma; bilateralcortical nodular hyperplasia or diagnosis of metastatic or primary adrenocortical carcinoma;

detection of residual masses, staging of tumors and follow-up; and

differentiation between tumors not originating from adrenal cortex.

In accordance with yet another feature of the invention, there is provided a method for functional adrenal scintigraphy and diagnosis of associated disease, wherein said associated conditions are selected from the group presenting with incidentaloma, or hormonally silent adenoma, wherein the adrenal-derived tumor is not anatomically confined to the adrenal glands.

In accordance with an added feature of the invention, there is provided a method of administering a compound, wherein positron-emission tomography (PET) is effective in detecting lesions of adrenocortical origin, residual masses; facilitating staging of tumors and follow-up. The associated conditions are selected from the group presenting with incidentaloma, adrenocortical adenoma, and adrenal tumors.

In accordance with yet another feature of the invention, there is provided a method of parenteral application of a compound defined in claim 5, wherein the radioactive halogen is selected from beta-emitting nuclides (¹³¹I, ⁸²Br) or alpha-emitting astatine (²¹¹At) for the purpose of radionuclide therapy of adrenocortical or extraadrenal malignancy.

FIG. 1 is a table showing the effect of structural changes of etomidate derivatives, and other Inhibitors by the displacement of ¹³¹I-IMTO binding.

Derivatives of etomidate displaced the radioligand ¹³¹I-IMTO with high potencies, except the (S)-enantiomer, which showed low binding affinity. The different esters (ETO, MTO, and FETO) showed similar potencies. The free acid is inactive. Substitution of the phenyl ring with iodine (4-Iodo-MTO) showed a slight effect, nevertheless, ¹³¹I-IMTO performed as a valuable radioligand in the displacement studies. Metyrapone and ketoconazole showed considerably lower binding affinities.

The available radiotracers for imaging the adrenal cortex and adrenal cortex-derived tumors are labeled cholesterol derivatives. These include 6β-[¹³¹I]-iodomethyl-19-norcholesterol (NP-59) (Basmadjian, 1975) and 6β-[⁷⁵Se]-selenomethyl-19-norcholesterol (Scintadren™) (Sakar, 1976). Both NP-59 and Scintadren™ accumulate in the adrenals slowly, within days, requiring long-lived radionuclides as a label (Iodine-131 T_{1/2}=8.04 days; Selen-75 T_{1/2}=120 days). Iodine-131 is also emitting beta-radiation, which contributes considerably to the radiation exposure. The diagnostic use of beta-emitters is no longer state of the art.

In view of the drawbacks of above mentioned agents with respect to patient care (high radiation exposure, repeated imaging procedures) the radiolabelled derivatives of etomidate and metomidate have greatly improved radionuclide imaging procedures for the detection and follow-up of adrenal disease.

The invention disclosed herein concerns radioactive compounds with high selectivity for adrenocortical tissue, providing metomidate labelled with a SPECT or PET radionuclide. ¹²³I-metomidate offers optimal imaging characteristics with SPECT, ¹⁸F-etomidate with PET. Labeled with a beta-emitting radionuclide (e.g., ¹³¹I), iodoetomidate, resp., iodometomidate may have potential for treatment of malignancy that shows increased uptake of the radiotracer. Therapeutic use in patients is based on high-affinity binding and slow release of said compounds, offering a sufficiently long residence time for delivering a therapeutic radiation dose.

Etomidate is known as a short-acting hypnotic with an adrenostatic side effect as a potent inhibitor of cortisol synthesis (Drake et al., 1998). High affinity binding has been demonstrated for the human mitochondrial cytochrome P450 enzymes CYP11B1 and CYP11B2, which catalyze the final steps in the biosynthesis of cortisol and aldosterone. Adrenal suppression has been observed with doses of 0.04 mg/kg (Diago et al. 1988), whereas the intravenous induction dose for anesthesia in adults is reported as 0.3 mg/kg. The high affinity of ETO derivatives as displacers of specific radioligand binding serves as a basis of an in vitro binding procedure. ¹³¹I-IMTO was used as a radioligand to characterize high affinity binding sites on crude membranes prepared from whole rat adrenals

Displacement of ¹³¹I-IMTO Binding

Compounds of formula I and derivatives were evaluated as competitive inhibitors of [¹³¹I]MTO binding. Test compounds were incubated at 0.01-100 nM concentrations. Non-specific binding was determined with etomidate (10 μM). The reaction was initiated by the addition of crude adrenal membranes and was terminated by filtration through Whatman GF/B filters (presoaked in buffer), followed by 2×4 mL washings with buffer. The filters containing membranes with bound radioligand were measured in a γ-spectrometer. IC₅₀ values (the molar concentration of compound necessary to inhibit binding by 50%) were determined for each test compound by non-linear, least squares regression analysis, using an iterative curve fitting routine.

The IC₅₀ values for selected derivatives of etomidate and metomidate are presented in FIG. 1. The ethyl ester (etomidate) shows the highest potency (IC₅₀=1.1 nM); the methyl ester (metomidate; IC₅₀=3.7 nM) and the 2-fluoroethyl ester (FETO; IC₅₀=3.0 nM) have similar potencies. The iodinated derivative, 4-iodo-MTO showed consistently a slightly higher value (IC₅₀=9.0 nM)). Moreover, it was demonstrated that (R)-configuration of the methyl substituent at the chiral C-atom is essential for binding, (S)-configuration is not tolerated (IC₅₀=492 nM); cleavage of the ester results in deactivation, the free acid (ETO-acid) is inactive (IC₅₀=123 μM); Metyrapone, a known, clinically used inhibitor, showed micromolar potency (IC₅₀=1.2 μM) when tested in this assay.

FIG. 2 is a graphic presentation showing the displacement of specific ¹³¹I-IMTO binding by ETO (circles), 4-I-ETO (squares), FETO (triangles), and metyrapol (diamonds).

A comparison of IC₅₀ values obtained by the displacement of specifically bound ¹³¹I-IMTO by structurally related compounds offered insight into the structural requirements for high-affinity binding in vivo.

In Vivo Evaluation of ¹³¹I-IMTO

Method: ¹³¹I-IMTO was used with a radiochemical purity >99% and a specific activity of 57 GBq/μmol. The radiotracer (0.5-1.1 MBq; 10-20 μCi) was injected into the tail vein of rats (female, 180-220 gram). Groups of four rats were sacrificed at specified times up to 24 hours post injection. The organs were excised and weighed, the radioactivity was measured at constant geometry using a γ-spectrometer with a NaI(Tl)-crystal. The data were expressed as percent of injected dose (ID) per organ and as percent of ID per gram tissue.

Results: ¹³¹I-IMTO showed high specific uptake in the adrenals of approximately 10% ID/g tissue with a radioactivity plateau exceeding 2 hours (FIG. 3). The radiotracer is primarily excreted by the kidneys. Renal activity is attributed to ¹³¹I-ETO-acid, which results from enzymatic cleavage of the methyl ester. The renal activity is increasing up to 4 hours post injection. Based on calculations of the target-to-non-target-ratios obtained in rats, the highest contrast for imaging of the adrenals is observed up to one hour post injection (FIG. 3). Based on these results, ¹³¹I-IMTO shows a high potential as a radiotracer for functional imaging of adrenal pathology. The biodistribution of ¹⁸F-FETO is shown in FIG. 4. In this case no renal accumulation of free acid is visible, because the free acid is not labelled.

FIG. 3 shows the distribution of radioactivity in organs after intravenous injection of ¹³¹I-IMTO in rats (means ±SD; n=4), followed up to 120 minutes post injection, expressed as a percentage of the injected dose per gram of organ weight.

FIG. 4 shows Target/Non-Target Ratios obtained with ¹³¹I-IMTO in rats

FIG. 5 shows the distribution of radioactivity in organs after intravenous injection of ¹⁸F-FETO in rats (means±SD; n=3), followed up to 60 minutes post injection, expressed as a percentage of the injected dose per gram of organ weight.

Clinical Application

Labelled etomidate derivatives have shown excellent characteristics as adrenal imaging agents. Metomidate binds selectively to tissue rich in P-450c11 enzyme activity, which is specifically expressed in adrenocortical tissue and tumors derived therefrom. High affinity binding to specific receptor sites requires high specific activity of the radiotracer, providing an adequate amount of radioactivity in a negligible mass of labelled derivative.

Safe application devoid of any pharmacological effect is dose related, therefore, trace amounts of the radiotracer are injected in a single intravenous dose. This is accomplished by carrier-free labelling of PET and SPECT radiotracers with high specific activities. Actually, the PET tracer ¹¹C-MTO is generally produced with a specific activity of 60 GBq/μmol, the SPECT tracer ¹²³I-IMTO is available with 100 GBq/μmol (2.7 Ci/μmol). External scintigraphy thus is performed with a microdose (0.6-5 μg) of the PET or SPECT tracer avoiding a hypnotic effect as well as cortisol suppression (SF=>2.800).

The acute toxicity of 4-iodometomidate was determined in mice. No dose-related mortality was observed by applying 2 microgram/kg body weight during a period of 14 days in mice (5 males and 5 females). Mice showed normal food intake and gained weight. None of the organs showed any pathological changes. The calculated dose would correspond to a 100-fold dose of ¹²³I-IMTO used in man.

Mutagenicity: The bacteriological test (Ames Test) using the same dosage (100 microgram/plate), showed no signs of a mutagenic effect.

Radiation exposure: The effective (whole body) dose was calculated as 0.0259 mSv/MBq. The effective dose in adults (70 kg) resulting from 185 MBq (5 mCi) of intravenously injected ¹²³I-IMTO for adrenal scintigraphy is approximately 4.8 mSv.

Patient dose: 185-220 MBq (5-6 mCi) of the SPECT radiotracer 370 MBq (10 mCi) of the PET radiotracer, injected as a bolus.

After the intravenous injection of the radiotracer, the scanning sequence is started. 14 frames are acquired during a total examination time of 45 minutes. No blood samples need to be taken. The study is evaluated with respect to tracer uptake in the adrenal lesion, in normal adrenals and in the liver. The imaging results are related to findings at surgery and biochemical screening.

Contrast for Imaging

Based on calculations of the target-to-non-target concentration ratios the highest contrast for imaging of the adrenals is observed up to 2 hours post injection (FIG. 3).

Advanced imaging techniques, i.e., ¹²³I-IMTO-SPECT or ¹⁸F-FETO-PET as well as SPECT/CT and PET/CT offer considerable advantages for the detection and differentiation of adrenal pathology:
Identification of hormonally silent adrenocortical adenoma (avoiding biopsy and biochemical screening);
Define nature of incidentaloma to exclude silent adrenal cortical or medullary dysfunction;
Detection of a primary adrenal cortical tumor/exclusion of any other origin;
Differential diagnosis of primary adrenocortical tumor and metastasis of other origin;
Discrimination between tumor originating from adrenal cortex and from adrenal medulla;
Discriminate between benign and malignant adrenocortical tumor to exclude adrenocortical dysfunction; metastatic or primary cortical cancer;
Visualize metastases or recurrence of adrenocortical cancer.

### BRIEF DESCRIPTION OF THE SEVERAL VIEWS OF THE DRAWING

FIG. 1: Displacement of ¹³¹I-IMTO binding by etomidate derivatives and other inhibitors (IC₅₀-values).

FIG. 2: Displacement of specific ¹³¹I-IMTO binding by ETO (circles), 4-I-ETO (squares), FETO (triangles), and metyrapol (diamonds) at increasing inhibitor concentrations.

FIG. 3: Distribution of radioactivity in organs up to 120 minutes after intravenous injection of ¹³¹I-IMTO in rats (means±SD; n=4), expressed as a percentage of the injected dose per gram of organ weight.

FIG. 4: Target/Non-Target Ratios obtained with ¹³¹I-Iodometomidate (IMTO) in rats.

FIG. 5: Distribution of radioactivity in organs up to 60 minutes after intravenous injection of ¹⁸F-FETO in rats (means±SD; n=3), expressed as a percentage of the injected dose per gram of organ weight.

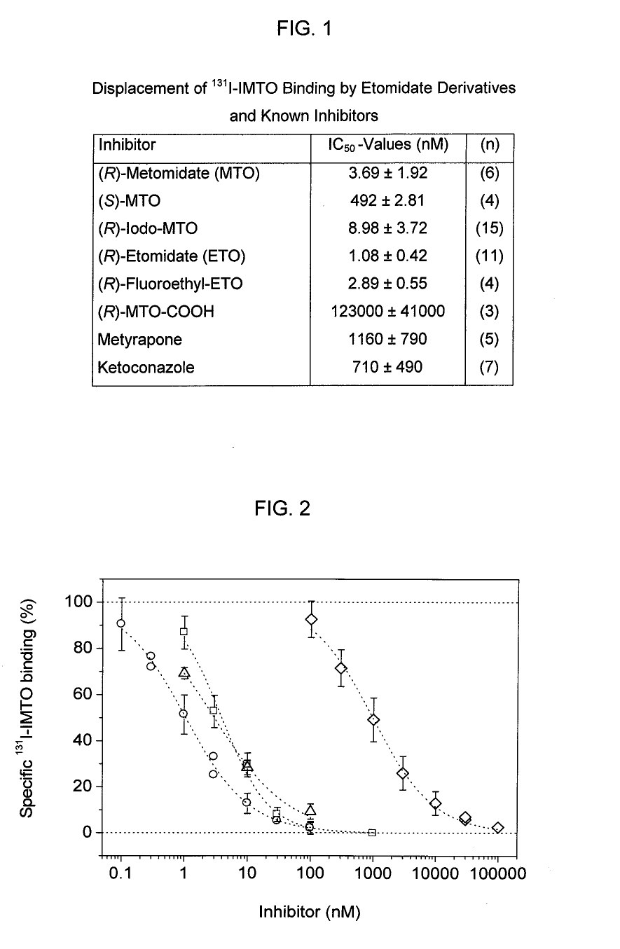

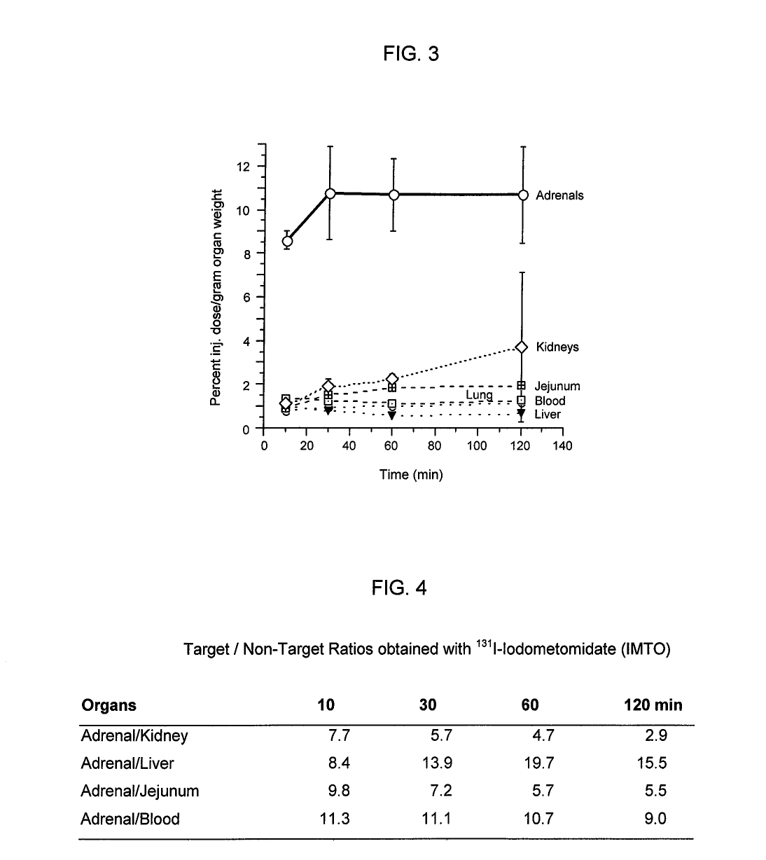

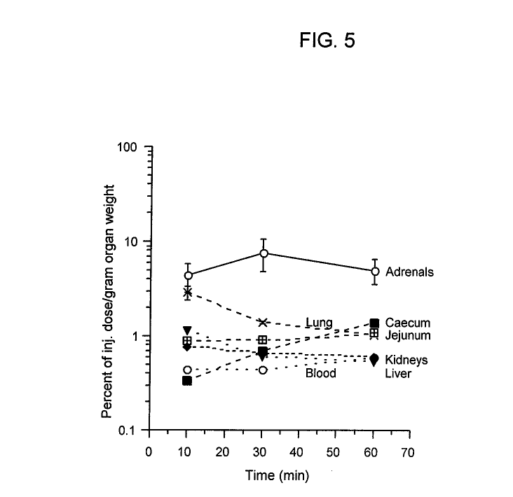

### DETAILED DESCRIPTION OF THE INVENTION

The present invention provides a compound of the general formula I:

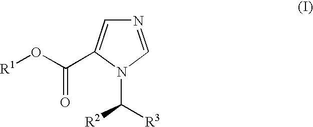

wherein
R¹ is linear or branched C₁-C₄ alkyl, and is optionally substituted with a halogen selected from the groups consisting of F, Cl, I or Br;
R² denotes an alkyl group containing 1 or 2 carbon atoms; and
R³ is phenyl, optionally substituted with a halogen;

As used herein, the expression “alkyl,” includes methyl and ethyl groups, and linear or branched propyl groups. Particular alkyl groups are methyl, ethyl, 2-fluoroethyl, n-propyl, and 2-propyl.

The term “halogen” as used herein, includes iodine, bromine, chlorine, fluorine, and astatine.

The substituent R¹ on the carboxylic ester group may be transformed to other substituents encompassed by the definition of R¹ by suitable reactions known in the art for the modification of carboxylic acid functions, i.e., by hydrolysis and esterification and/or transesterification. The starting materials for the preparation of the novel compounds of formula (I) are known or they have been obtained by enantioselective synthesis disclosed previously.

Particularly preferred novel compounds in accordance with the present invention are those ester compounds wherein R¹ is alkyl substituted with a halogen, preferably with a radioactive halogen.

The compound of formula (I) in accordance with the present invention is suitably a radiolabelled derivative of formula IA:

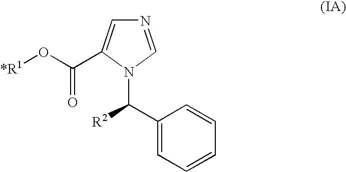

wherein
R¹ is linear or branched C₁-C₄ alkyl, and is optionally substituted with an alpha-halogen; said halogen being selected from fluorine, preferably radioactive fluorine;
R² denotes an alkyl group containing 1 or 2 carbon atoms.

Preferred are compounds of formula IA, wherein R¹ is 2-fluoroethyl and said halogen is ¹⁸F.

The compound of formula (I) in accordance with the present invention is suitably a compound wherein phenyl is substituted with a halogen of formula IB:

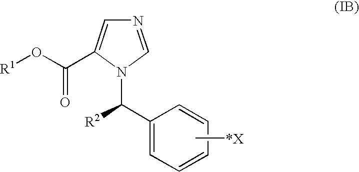

wherein
R¹ is linear or branched C₁-C₄ alkyl, and is optionally substituted with a halogen selected from the groups consisting of F, Cl, I or Br;
R² denotes an alkyl group containing 1 or 2 carbon atoms; and
R³ is phenyl, substituted with a radioactive halogen, selected from the group consisting of, ¹²³I, ¹²⁴I, ¹²⁵I ¹³¹I, ⁷⁶Br, ⁸²Br, ²¹¹At, or ¹⁸F;

Preferred are compounds of formula IB, wherein said halogen is ¹²³I, ¹²⁴I, ¹³¹I, or ¹⁸F.

The present invention is described below in more detail in connection with the synthesis of an R¹ derived labelled ester (R)-2-¹⁸F-fluoroethyl 1-(1-phenylethyl)-1H-imidazole-5-carboxylate and the R³ derived radiotracer (R)-1-[1-(4-¹³¹I-iodo-phenyl)ethyl]-1H-imidazole-5-carboxylic acid methyl ester (¹³¹I-IMTO). Examples are given merely for illustrative purposes and shall in no way be understood as a limitation of the scope of the present invention which is given by the patent claims.

### EXAMPLES

The substituent R¹ on the carboxylic ester group may be transformed to other substituents defined as R¹ by suitable reactions known in the art for the modification of carboxylic acid functions. However, introducing a positron emitter requires special techniques for the conversion of the radionuclide into a reactive alcohol for subsequent esterification, generally performed on-line using closed synthesis modules.

Synthesis of Modified Esters

Transesterification of commercially available etomidate at ambient temperature in dry MeOH, n-propanol, or 2-propanol in the presence of the corresponding sodium alkoxide yielded metomidate, and the n-propyl and 2-propyl esters, respectively.

### Example 1

### Synthesis of (R)-2-fluoroethyl 1-(1-phenylethyl)-1H-imidazole-5-carboxylate

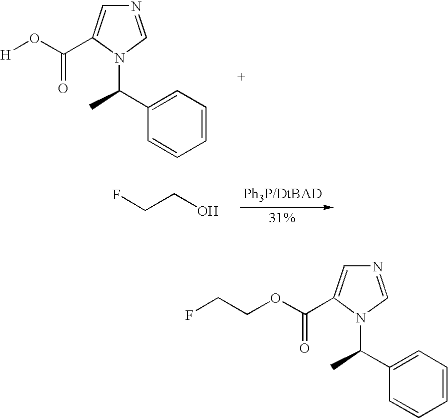

A solution of DtBAD (0.128 g, 0.554 mmol) in dry toluene (2 mL) was added to a stirred mixture of Ph₃P (0.145 g, 0.554 mmol), methyl 1H-imidazole-5-carboxylate (0.100 g, 0.462 mmol) and 2-fluoroethanol (44 mg, 0.040 mL, 0.681 mmol, handle with care!) in dry toluene (2 mL) under an atmosphere of argon. After 18 h, water (two drops) was added and the mixture was concentrated under reduced pressure to give a residue which was purified by flash chromatography (first column: 60 g of silica gel, hexane/Et₂O/iPr₂NH 5/10/1, R_{f} 0.25, 98 mg of mixture of 2-fluoroethyl ester and hydrazo ester; second flash chromatography: 40 g silica gel, Et₂O as eluent, R_{f} 0.30) to give the product (38 mg, 31%) as a crystalline solid, mp 51° C. (hexane); [α]²⁰_{D}=+106.29 (c 0.72, acetone). Anal. (C₁₄H₁₅FN₂O₂) C, H, N.

### Radiosynthesis of (R)-2-¹⁸F-fluoroethyl 1-(1-phenylethyl)-1H-imidazole-5-carboxylate (¹⁸F-FETO)

Synthesis is based on the nucleophilic radiofluorination with no-carrier-added ¹⁸F-fluoride after kryptofix 2.2.2.-activated nucleophilic substitution of 1,2-dibromoethane in acetonitrile to yield 2-¹⁸F-fluoroethyl bromide for ¹⁸F-fluoroethylation of (R)-1-(1-phenylethyl)-1H-imidazole-5-carboxylic acid as the tetrabutylammonium salt to yield the labeled fluoroethyl derivative. The radioligand is produced with a specific activity of approx. 40 GBq/μmol (1.1 Ci/μmol).

The substituent R³ is labelled by oxidative destannylation of especially synthesized precursors, which facilitate rapid labelling under mild reaction conditions. Therefore, 4-iodo-metomidate or 4-iodo-etomidate, respectively, is converted to the 4-trimethylstannyl derivative to serve as a precursor for labelling metomidate and etomidate with any radiohalogen.

### Radiosynthesis of 4-¹²³1-iodophenyl-metomidate (¹²³I-IMTO)

Radiohalogenated compounds of formula IB are conveniently prepared by reacting a stannylated precursor with radiohalogen (Iodine-123; iodine-131; bromine-76 and others) in the presence of an oxidizing agent, at room temperature. The radioligand ¹³¹I-IMTO is produced with a specific activity of >50 GBq/μmol, resp. >1.35 Ci/μmol.

Substitution with a radiohalogen in the phenyl ring offers access to diagnostic as well as therapeutic MTO-derivatives. Radionuclides for therapy are beta- and alpha-emitting halogens, e.g., ¹³¹I, ⁸²Br, and ²¹¹At.

While the invention has been described in its preferred form or embodiment with some degree of particularity, it is understood that this description has been given only by way of example and that numerous changes in the details of synthesis, fabrication, and use, including the combination and arrangement of parts, may be made without departing from the spirit and scope of the invention.

### LITERATURE RELATED TO THE PRIOR ART

Basmadjian G P, Hetzel K R, Ice R D, Beierwaltes W H (1975) Synthesis of a new adrenal cortex imaging agent 6□-[¹³¹I]-iodomethyl-19-norcholest-5(10)en-3□-ol (NP-59). J. Labelled Compd. & Radiopharm. XI: 427-434.
Sakar S D, Ice R D, Beierwaltes W H, Gill S P, Balanchandran S, Basmadjian G P (1976) Selenium-75-19-selenocholesterol—a new adrenal scanning agent with high concentration in the adrenal cortex. J Nucl Med 17: 212-217.
W. H. Beierwaltes, D. M. Wieland, R. D. Ice, J. E. Seabold, S. D. Sarkar, S. P. Gill, and S. T. Mosley: Localization of radiolabeled enzyme inhibitors in the adrenal gland. J. Nucl. Med., 17(11), 998-1002, 1976.
W. H. Beierwaltes, D. M. Wieland, S. T. Mosley, D. P. Swanson, S. D. Sarkar, J. E. Freitas, J. H Thrall, and K. R. Herwig: Imaging the adrenal glands with radiolabeled inhibitors of enzymes: concise communication. J. Nucl. Med., 19(2), 200-203, 1978.
Wu J. L., Wieland D. M., Beierwaltes W. H., Swanson D. P., Brown L. E.: Radiolabelled enzyme inhibitors—enhanced localization following enantiomeric purification. J. Labelled Compd. & Radiopharm., XVI(1), 6-9, 1979.
Wieland D M: Radiolabeled enzyme inhibitors—Adrenocortical enzymes. In: Receptor-binding radiotracers, Vol. 1, 127-146, Ed. W. C. Eckelman, Chemical Rubber Co. Press, Cleveland, Ohio, 1982.
Robien W. and Zolle I. (1983) Synthesis of radioiodinated metyrapone—A potential agent for functional imaging of the adrenal cortex. Int. J. Appl. Radiat. Isot. 34: 907-914.
Zolle, W. Woloszczuk, and R. Höfer: Synthesis and in vitro evaluation of metyrapone derivatives as potential inhibitors of 11□-hydroxylase activity. In: Radiopharmaceuticals and labelled compounds, 337-342, IAEA-CN-45/67, Vienna, 1985.
Yu, J., Zolle, I., Mertens, J., and Rakias, F.: Synthesis of 2-[¹³¹I]-iodophenyl-metyrapone using Cu(I)-assisted nucleophilic exchange labelling: Study of the reaction conditions. Nucl. Med. & Biol. 22(2): 257-262 (1995).
Vanden Bossche H, Willemsens G, Cools W, Bellens D (1984) Effects of etomidate on steroid biosynthesis in subcellular fractions of bovine adrenals. Biochemical Pharmacology: 33(23), 3861-3868.
Belelli, D.; Lambert, J. J.; Peters, J. A.; Wafford, K.; Whiting, P. J. (1997) The interaction of the general anesthetic etomidate with the g-aminobutyric acid type A receptor is influenced by a single amino acid. Proc. Natl. Acad. Sci. USA 94, 11031-11036.
Franks, N. P. (2006) Molecular targets underlying general anaesthesia. Br. J. Pharmacol. 147 Suppl 1, S72-81.
Engelhardt D (1994) Steroid biosynthesis inhibitors in Cushing's syndrome. Clin Investig 72: 481-488.
Weber M M, Lang J, Abedinpour F, Zeilberger K, Adelmann B, Engelhardt D (1993) Different inhibitory effect of etomidate and ketoconazole on the human adrenal steroid biosynthesis. Clin. Invest. 71: 933-938.
Godefroi, E. F., Janssen, P. A. J., Van der Eycken, C. A. M., Van Heertum, A. H. M. T., Niemegeers, C. J. E. (1965) DL-1-(1-Arylalkyl)imidazole-5-caroxylate esters. A novel type of hypnotic agents. J. Med. Chem. 8: 220-223.
Synthesis of etomidate U.S. Pat. No. 3,354,173 issued Nov. 21, 1967. Expired Nov. 1984.

### Labelled Metomidate

Bergström, M.; Bonasera, T. A.; Lu, L.; Bergström, E.; Backlin, C.; Juhlin, C.; Långström, B. (1998) In vitro and in vivo primate evaluation of carbon-11-etomidate and carbon-11-metomidate as potential tracers for PET imaging of the adrenal cortex and its tumors. J. Nucl. Med. 39, 982-989.
Bergström, M.; Juhlin, C.; Bonasera, T. A.; Sundin, A.; Rastad, J.; Åkerström, G.; Långström, B. (2000) PET imaging of adrenal cortical tumors with the 11β-hydroxylase tracer ¹¹C-metomidate. J. Nucl. Med. 41, 275-282.
Wadsak, W., Mitterhauser, M. (2003) Synthesis of [¹⁸F]FETO, a novel potential 11β-hydroxylase inhibitor. J. Label. Compds. Radiopharm. 46, 379-388.
Mitterhauser, M.; Wadsak, W.; Wabnegger, L.; Sieghart, W.; Viernstein, H.; Kletter, K.; Dudczak, R. (2003) In vivo and in vitro evaluation of ¹⁸F-FETO with respect to the adrenocortical and GABAergic system in rats. Eur. J. Nucl. Med. & Molec. Imaging 30, 1398-1401.
Schirbel, A.; Zolle, I.; Hammerschmidt, F.; Berger, M. L.; Schiller, D.; Kvaternik, H.; Reiners, Chr. (2004) [^{123/131}I]Iodometomidate as a radioligand for functional diagnosis of adrenal disease: synthesis, structural requirements and biodistribution. Radiochim. Acta 92: 297-303.
Hammerschmidt, F.; Peric Simov, B.; Schmidt, S.; Schneider, S.; Zolle, I. (2005) Chemoenzymatic synthesis of stannylated metomidate as a precursor for electrophilic radiohalogenations—regioselective alkylation of methyl 1H-imidazole-5-carboxylate. Monatshefte Chem. 136, 229-239.
Wadsak, W.; Mitterhauser, M.; Rendl, G.; Schuetz, M.; Mien, L. K.; Ettlinger, D. E.; Dudczak, R.; Kletter, K.; Karanikas, G. (2006) ¹⁸F-FETO for adrenocortical PET imaging: a pilot study in healthy volunteers. Eur. J. Nucl. Med. Mol. Imaging 33, 669-672.
Zolle I. M., Berger M. L., Hammerschmidt F., Hahner S., Schirbel A., Peric-Simov B. (2008) New selective inhibitors of steroid 11b-hydroxylation in the adrenal cortex—Synthesis and SAR of potent etomidate analogues. J. Med. Chem. (accepted for publication)

### Literature on Incidentalomas

Siren J E, Haapiainen R K, Huikuri K T, et al. (1993) Incidentalomas of the adrenal gland: 36 operated patients and review of literature. World J. Surg. 17: 634-639.
Reincke M, Fassnacht M, Väth S, Mora P, Allolio B (1996) Adrenal incidentalomas: A manifestation of the metabolic syndrome. Endocrine Research 22(4): 757-761.
Abecasis M, McLoughlin M J, Lange B, Kudlaw J E (1985) Serendipitous adrenal masses: Prevalence, significance and management. Am. J. Surg. 149: 783.
Herrera M F, Grant C S, van Heerden J A, et al. (1991) Incidentally discovered adrenal tumors: an institutional perspective. Surgery 110: 1014-1021.
Kloos R T, Gross M D, Francis I R, Korobkin M, Shapiro B (1995) Incidentally discovered adrenal masses. Endocrin Rev. 16: 460-484.
   Lit. Clinical Experience with ¹¹C-metomidate
Eriksson, B.; Bergstrom, M.; Sundin, A.; Juhlin, C.; Örlefors, H.; Öberg, K.; Långström, B. (2002) The role of PET in localization of neuroendocrine and adrenocortical tumors. Ann. N.Y. Acad. Sci. 970, 159-169.
Khan, T. S.; Sundin, A.; Juhlin, C.; Långström, B.; Bergström, M.; Eriksson, B. (2003) ¹¹C-metomidate PET imaging of adrenocortical cancer. Eur. J. Nucl. Med. & Molec. Imaging 30, 403-410.
Minn, H.; Salonen, A.; Friberg, J.; Roivainen, A.; Viljanen, T.; Långsjö, J.; Salmi, J.; Välimäki, M.; Någren, K.; Nuutila, P. (2004) Imaging of adrenal incidentalomas with PET using ¹¹C-metomidate and ¹⁸F-FDG. J. Nucl. Med. 45, 972-979.
Hennings, J.; Lindhe, Ö.; Bergström, M.; Långström, B.; Sundin, A.; Hellman, P. (2006) ¹¹C-metomidate positron emission tomography of adrenocortical tumors in correlation with histopathological findings. J. Clin. Endocrinol. Metab. 91, 1410-1414.
   Literature Related to Toxic Effect of Etomidate when Used as a Hypnotic
Drake W M, Perry L A, Hinds C J, Lowe D G, Reznek R H, Besser G M (1998) Emergency and prolonged use of intravenous etomidate to control hypercortisolemia in a patient with Cushing's syndrome and peritonitis. J. Clin. Endocrinol. Metab. 83: 3542-3544.
Ledingham I, Watt I (1983) Influence of sedation on mortality in critically ill multiple trauma patients. Lancet, Jun. 4, 1270.
Fellows, I. W.; Bastow, M. D.; Byrne, A. J.; Allison, S. P. (1983) Adrenocortical suppression in multiply injured patients: a complication of etomidate treatment. Brit. Med. J. 287: 1835-1837.
Fellows I. W.; Byrne A. J.; Allison S. P. (1983) Adrenocortical suppression with etomidate. The Lancet, 54-55.
Allolio, B., Stuttmann, R., Fischer, H., Leonhard, W., Winkelman, W. (1983) Long-term etomidate and adrenocortical suppression. The Lancet ii: 626.
Wagner R L, White P F, Kan P B, Rosenthal M H, Feldman D (1984) Inhibition of adrenal steroidogenesis by the anesthetic etomidate. N. Engl. J. Med. 310: 1415-1421.
Diago, M. C.; Amado, J. A.; Otero, M.; Lopez-Cordovilla, J. J. (1988) Anti-adrenal action of subanaesthetic dose of etomidate. Anaesthesia 43, 644-645.

### Lit. for Fluoroethylation

Wester H J, Herz M, Weber W, Heiss P, Senekowitsch-Schmidtke R, Schwaiger M, Stöcklin G (1999) Synthesis and radiopharmacology of O-(2-¹⁸F-fluoroethyl)-L-tyrosine for tumor imaging. J. Nucl. Med. 40: 205-212.
Wester H-J, Willoch F, Tölle TR, Munz F, Herz M, Øye I, Schadrack J, Schwaiger M, Bartenstein P (2000) 6-O-(2-[¹⁸F]fluoroethyl)-6-O-desmethyldiprenorphine (¹⁸F]DPN): Synthesis, biologic evaluation, and comparison with [¹¹C]DPN in humans. J Nucl Med 41: 1279-1286.
Hamacher, K., Coenen, H. H. (2002) Efficient routine production of the ¹⁸F-labelled amino acid O-(2-¹⁸F-fluoroethyl)-L-tyrosine. Appl. Radiat. Isot. 57: 853-856.
Wadsak, W., Mitterhauser, M., Zolle, I. (2002) Synthesis of [¹⁸F]FETO, a novel PET-tracer for adrenal scintigraphy. Eur. J. Nucl. Med. & Molec. Imaging Vol. 29: Suppl. 1, S59 (Abstract).
Wadsak W, Mitterhauser M (2003) Synthesis of [¹⁸F]FETO, a novel potential 11b-hydroxylase inhibitor. J. Lab. Compd. Radiopharm. 46: 379-388.
Mitterhauser, M.; Wadsak, W.; Wabnegger, L.; Sieghart, W.; Viernstein, H.; Kletter, K.; Dudczak, R. (2003) In vivo and in vitro evaluation of ¹⁸F-FETO with respect to the adrenocortical and GABAergic system in rats. Eur. J. Nucl. Med. & Molec. Imaging 30, 1398-1401.

1. A compound of the general formula (I) 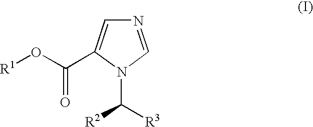
wherein
R¹ is linear or branched C₁-C₄ alkyl, and is optionally substituted with a halogen selected from the groups consisting of F, Cl, I or Br;
R² denotes an alkyl group containing 1 or 2 carbon atoms; and
R³ is phenyl, optionally substituted with a halogen;

2. The compound of formula (I), wherein the ester is substituted with halogen, suitably as a radiolabeled ester derivative of formula IA: 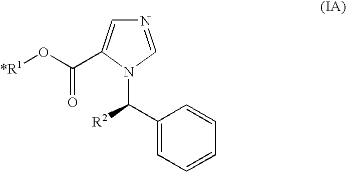
wherein
R¹ is linear or branched C₁-C₄ alkyl, and is optionally substituted with an alpha-halogen; said halogen being radioactive;
R² denotes an alkyl group containing 1 or 2 carbon atoms.

3. The compound of claim 2, wherein R¹ is radioactive 2-fluoroethyl, R² is methyl and R³ is phenyl; wherein the compound is ¹⁸F-etomidate (18F-FETO);

4. Methods for clinical application of 1-(1-arylalkyl)-1H-imidazole-5-carboxylate ester derivatives of formula (I) with modified functionality R¹, R², and R³, incorporating a radioactive halogen, wherein the compound is either prepared shortly prior to administering to the subject, or prepared at least one day before the imaging is performed, and stored until needed.

5. A method for performing adrenal scintigraphy for the diagnosis of associated disease, the method comprising: (a) administering to a patient an effective amount of radioactivity of a compound defined in claim 2 or 3 wherein R¹ is radioactive 2-fluoroethyl; or a compound wherein R³ is phenyl, substituted with a radioactive halogen of the following formula: 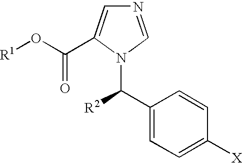
wherein R¹ and R² are each methyl, and X is radioactive iodine and wherein the compound is *I-metomidate (IMTO); or wherein R¹ is ethyl, R² is methyl, and X is radioactive iodine, wherein the compound is *I-etomidate (1-ETO), wherein the radioactive halogen is selected from the group consisting of ¹²³I, ¹²⁴I, ¹²⁵I, ¹³¹I, ⁷⁶Br, and ¹⁸F; (b) applying a suitable tomographic procedure, i.e., SPECT or PET.

6. A method for adrenal scintigraphy for the localization and characterization of abnormal adrenocortical function, wherein said associated conditions are Cushing's syndrome; primary aldosteronism; and the incidentally discovered adrenal mass; especially, adenoma; bilateralcortical nodular hyperplasia; adrenocortical carcinoma; and hormonally silent adenoma.

7. A method for functional adrenal scintigraphy and diagnosis of associated disease, wherein said associated conditions are selected from the group of conditions presenting with hyperfunctioning adrenal(s), adrenocortical adenoma, and adrenal tumors, said method comprising:
a. diagnosis of adrenocortical adenoma, bilateralcortical nodular hyperplasia, or diagnosis of metastatic or primary adrenocortical carcinoma in patients;
b. detection of residual masses, staging of tumors and follow-up; and
c. differentiation between tumors not originating from adrenal cortex.

8. A method for functional adrenal scintigraphy and diagnosis of associated disease, wherein said associated conditions are selected from the group presenting with incidentaloma, or hormonally silent adenoma, wherein the adrenal-derived tumor is not anatomically confined to the adrenal glands.

9. The method of administering a compound defined in claim 2, wherein positron-emission tomography (PET) is effective in detecting lesions of adrenocortical origin, residual masses; facilitating staging of tumors and follow-up. The associated conditions are selected from the group presenting with incidentaloma, adrenocortical adenoma, and adrenal tumors.

10. A method of parenteral application of a compound defined in claim 5, wherein the radioactive halogen is selected from beta-emitting nuclides (¹³¹I, ⁸²Br) or alpha-emitting astatine (²¹¹At) for the purpose of radionuclide therapy of adrenocortical or extraadrenal malignancy.
